(19) United States
(12) Patent Application Publication
(10) Pub. No.: US 2026/0061071 A1
(43) Pub. Date: Mar. 5, 2026

(54) Multi-Functional Nanoparticle System for Delivery of NAD+ Precursors, Sirtuin Activators, Senolytic Agents, and Stem Cells with pH-Responsive Release

(71) Applicant: Perron Shimizu, Anchorage, AK (US)

(72) Inventors: Perron Shimizu, Anchorage, AK (US); John Dunlop, San Antonio, TX (US)

(21) Appl. No.: 19/309,646

(22) Filed: Aug. 26, 2025

Related U.S. Application Data
(60) Provisional application No. 63/690,758, filed on Sep. 4, 2024.

(51) Int. Cl. A61K 47/69; A61K 9/00; A61K 31/352; A61K 31/506; A61K 31/5377; A61K 31/706; A61K 49/18

(52) U.S. Cl. CPC A61K 47/6937; A61K 9/0019; A61K 31/352; A61K 31/506; A61K 31/5377; A61K 31/706; A61K 47/6901; A61K 49/1878

(57) ABSTRACT

Disclosed is a multi-functional nanoparticle delivery system comprising a biodegradable core of poly(lactic-co-glycolic acid) (PLGA) or calcium phosphate (CaP), encapsulating a nicotinamide adenine dinucleotide (NAD⁺) precursor and a sirtuin activator. Surrounding the core is a liposomal or polymeric layer containing one or more senolytic agents, and an outer layer of magnetic iron oxide nanoparticles for targeted delivery, external manipulation, and imaging. In some embodiments, the nanoparticle surface is functionalized for conjugation with autologous mesenchymal stem cells to enhance homing and regenerative potential. The system is pH-responsive, releasing its payload in acidic microenvironments typical of senescent or diseased cells, while remaining stable at physiological pH. This integrated design supports NAD⁺ restoration, sirtuin activation, senescent cell clearance, and tissue regeneration. Applications include treatment of age-related diseases, regenerative medicine, cardiovascular and neurodegenerative disorders, and cosmetic skin rejuvenation.

### CROSS-REFERENCE TO RELATED APPLICATIONS

This application claims the benefit of U.S. Provisional Application No. 63/690,758, filed Sep. 4, 2024, the entire disclosure of which is incorporated herein by reference.

### FIELD OF THE INVENTION

The present invention relates to a multi-functional anti-aging nanoparticle drug delivery system. More particularly, it pertains to nanoparticle formulations incorporating NAD⁺ precursors, sirtuin activators, senolytic agents, and magnetic targeting components, optionally combined with autologous mesenchymal stem cells (MSCs) for targeted rejuvenation and tissue regeneration.

### BACKGROUND OF THE INVENTION

Age-related disorders are characterized by progressive loss of cellular function, depletion of nicotinamide adenine dinucleotide (NAD⁺) , impaired sirtuin activity, and the accumulation of senescent cells. Senescent cells secrete pro-inflammatory and matrix-degrading factors that contribute to tissue dysfunction, skin aging, metabolic decline, neurodegeneration, and cardiovascular disease.

Existing approaches to counter aging and related conditions include oral supplementation with NAD⁺ precursors, small molecule sirtuin activators, and senolytic compounds that selectively remove senescent cells. However, these strategies suffer from limitations such as poor bioavailability, rapid clearance, off-target toxicity, and lack of controlled release. In particular, senolytic agents such as dasatinib and quercetin require targeted delivery to minimize systemic side effects, while NAD⁺ precursors and sirtuin activators require stable encapsulation to reach intracellular targets effectively.

Nanoparticle delivery systems have been explored to improve stability and targeting, but most existing designs focus on single-agent delivery and lack multi-functional integration. There remains a need for an integrated nanoparticle platform capable of simultaneously restoring NAD⁺ levels, enhancing sirtuin activity, and clearing senescent cells with pH-sensitive controlled release, while also enabling combination with autologous stem cells for regenerative therapy.

Current nanoparticle systems are limited either to polymeric carriers such as PLGA or mineral carriers such as calcium phosphate (CaP). The present invention overcomes these limitations by providing a flexible platform in which the nanoparticle core may be either PLGA or CaP, depending on therapeutic requirements.

### SUMMARY OF THE INVENTION

The present invention provides a multi-functional nanoparticle delivery system designed for the treatment of age-related disorders and for use in regenerative medicine. The system comprises:
(i) a biodegradable nanoparticle core, which in certain embodiments is poly(lactic-co-glycolic acid) (PLGA) and in other embodiments is calcium phosphate (CaP), encapsulating a nicotinamide adenine dinucleotide (NAD⁺) precursor (such as nicotinamide riboside, NR) and a sirtuin activator (such as SRT2104);
(ii) an intermediate liposomal or polymeric layer containing one or more senolytic agents, including but not limited to dasatinib and quercetin, configured for pH-responsive release; and
(iii) an outer layer of magnetic iron oxide nanoparticles configured to facilitate targeted delivery, external magnetic manipulation, and optional imaging.

In certain embodiments, the nanoparticle surface is further functionalized to enable conjugation with autologous mesenchymal stem cells (MSCs), thereby enhancing tissue targeting and regenerative potential. The system is pH-responsive, providing controlled release of its encapsulated agents under acidic conditions typical of senescent or diseased cellular environments, while maintaining stability at physiological pH.

This integrated design simultaneously restores NAD⁺ levels, activates sirtuin pathways, selectively clears senescent cells, and promotes tissue regeneration. Applications include, but are not limited to, the prevention and treatment of age-associated conditions such as skin aging, osteoarthritis, metabolic dysfunction, neurodegenerative disorders, and cardiovascular disease, as well as cosmetic and regenerative therapies.

### BRIEF DESCRIPTION OF THE DRAWINGS

FIG. 1 is a schematic illustration of the multi-layer nanoparticle system (100), including the polymeric PLGA core (110) encapsulating nicotinamide riboside (NR) and SRT2104, the pH-sensitive liposomal middle layer (120) carrying senolytic agents dasatinib and quercetin, and the iron oxide outer layer (150) enabling magnetic targeting and imaging.

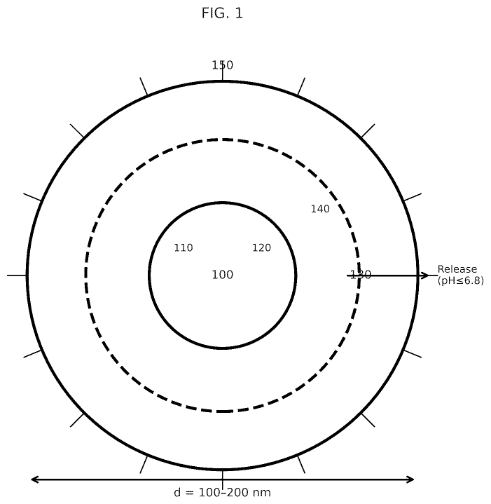

FIG. 2 depicts the release kinetics of encapsulated agents at varying pH values, where curve 300 represents release at physiological pH 7.4, curve 305 represents release under mildly acidic conditions (pH 6.8), and curve 310 represents release in lysosomal/strongly acidic conditions (pH 6.0).

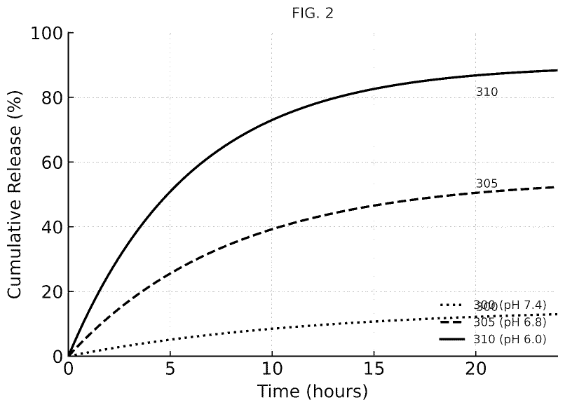

FIG. 3 shows representative transmission electron microscopy (TEM) images of the nanoparticles, highlighting the layered architecture (130) and the presence of the iron oxide surface layer (150) that provides magnetic responsiveness and structural stability.

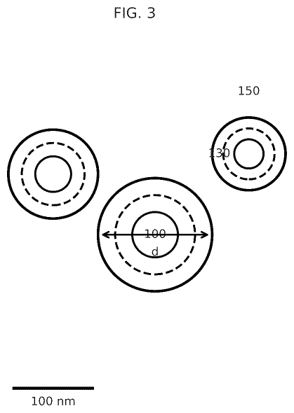

FIG. 4 illustrates mesenchymal stem cell (MSC) conjugation, in which the MSC (200) is coated with a protective polymer layer (205) and tethered to nanoparticles (230) via bio-orthogonal linker molecules (210) at attachment points (220).

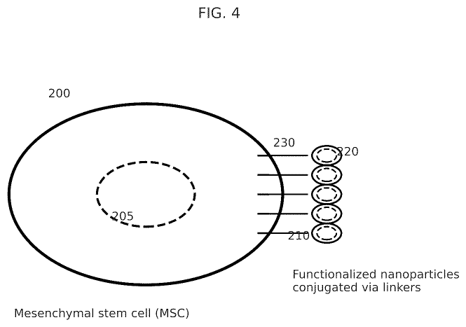

FIG. 5 presents experimental results demonstrating NAD⁺ restoration and sirtuin activation, where bar 400 represents relative intracellular NAD⁺ levels and bar 410 represents relative SIRT1 activity in treated versus control tissues.

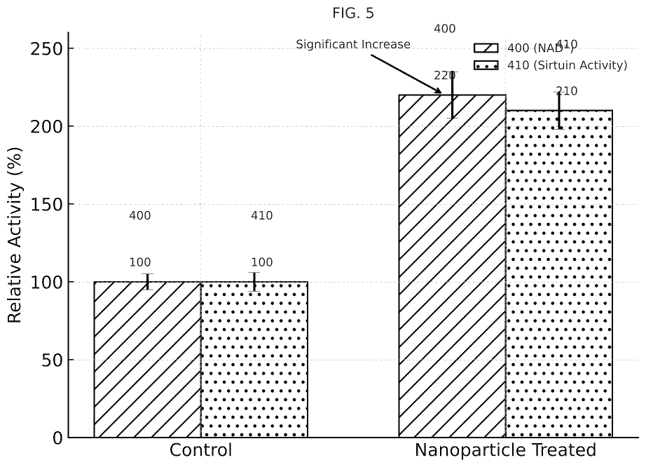

FIG. 6 shows senescent cell clearance and tissue regeneration outcomes, where bar 500 indicates reduction in senescent cell burden following treatment, and bar 510 represents increases in tissue regeneration markers such as collagen deposition and proliferative index.

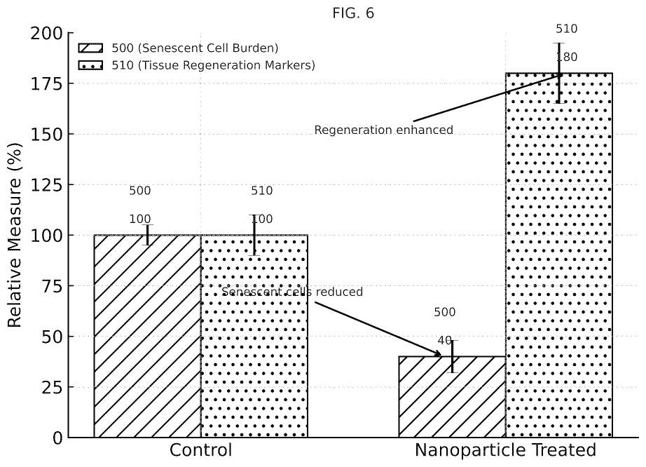

### DESCRIPTION OF INVENTION

The following description provides exemplary embodiments, compositions, architectures, and methods of use for a multi-functional anti-aging nanoparticle delivery system. Unless otherwise indicated, the singular forms include the plural forms, and the term “comprise” or “may comprise”refers to the inclusion of stated elements without excluding additional elements.

In a first embodiment, the therapeutic system is delivered as a multi-layer nanoparticle that carries small-molecule drugs and optionally living cells. The nanoparticle comprises a polymeric core, a liposomal intermediate layer, and an iron oxide outer layer, optimized for sequential release and targeted delivery.
Core Layer—In certain embodiments, the core is PLGA; in other embodiments, it is CaP. Both options provide pH-sensitive degradation and controlled release, though CaP additionally mimics bone mineral composition and enhances intracellular delivery. PLGA Nanoparticle (NAD⁺ Precursors & Sirtuin Activators): The innermost core may comprise a biodegradable polymer such as poly(lactic-co-glycolic acid) (PLGA). Nicotinamide riboside (NR), a precursor to NAD⁺, and SRT2104, a sirtuin activator, are embedded within this PLGA core. The degradation rate of PLGA is tunable by adjusting the lactide: glycolide ratio, providing controlled release. The nanoparticles are typically ˜100 nm in diameter. As PLGA hydrolyzes under physiological conditions, NR and SRT2104 are released in a controlled manner to restore NAD⁺ levels and activate sirtuins. Encapsulation improves stability and pharmacokinetics of the compounds.
Middle Layer—Liposomal Coat (Senolytic Agents with pH-Sensitive Release): The polymeric core may be surrounded by a phospholipid liposomal layer containing senolytic agents such as dasatinib and quercetin. The liposomal bilayer is engineered to destabilize in acidic environments (pH≤6.5), thereby releasing senolytics selectively at sites of senescence. At physiological pH (7.4), the liposome remains intact. PEGylation may be used to enhance circulation time. This layer provides targeted senolytic activity while minimizing systemic exposure.
Outer Layer—Iron Oxide Nanoparticles (Magnetic Targeting and Shielding): The outermost layer may include superparamagnetic iron oxide nanoparticles (SPIONs). These confer responsiveness to external magnetic fields for targeted guidance and enable magnetic resonance imaging. SPIONs are typically 3-7 wt % of total weight and are coated with biocompatible materials. This layer enhances stability and enables monitoring of biodistribution.
Autologous MSC Integration—Cell Attachment and Protective Coating: In some embodiments, nanoparticles are attached to autologous mesenchymal stem cells (MSCs). MSCs may be coated with a biocompatible polymer to protect viability, and nanoparticles are tethered using cleavable linkers. This allows MSCs to act as living carriers, homing to damaged tissue, while nanoparticles detach and release therapeutic agents locally.

In an alternative embodiment, the nanoparticle system employs a calcium phosphate (CaP)-based pH-responsive nanocarrier rather than PLGA. CaP is bioresorbable and mimics natural bone composition, offering enhanced biocompatibility and pH-sensitive dissolution in acidic environments.
Core Layer—Calcium Phosphate Nanoparticles: In this embodiment, the core comprises calcium phosphate encapsulating therapeutic agents such as nicotinamide riboside, SRT2104, and optionally methylene blue. CaP dissolves preferentially under acidic conditions, enabling endosomal release. Particle diameters are typically 80-140 nm with polydispersity indices of ≤0.20.
Intermediate Shell—Liposomal Coating: As in the PLGA-based design, a liposomal shell may be applied to encapsulate senolytic agents such as dasatinib and quercetin. The liposome is engineered to destabilize in acidic environments, triggering selective senolytic release.
Outer Surface Functionalization: The outer surface may include magnetic iron oxide nanoparticles or gold nanoparticles for magnetic or photothermal targeting. Targeting ligands such as peptides or antibodies may be conjugated to enhance localization.
MSC Integration: As with the polymeric design, autologous MSCs may be used as delivery vehicles. CaP nanoparticle attachment does not compromise viability when a protective coating is employed. MSCs serve to home to inflamed or senescent tissues, enhancing targeted delivery and regenerative outcomes.
Mechanism of Action: Following intravenous administration, MSCs carrying nanoparticles are guided to senescent or damaged tissues via magnetic and biological targeting. At the target site, cleavable linkers detach nanoparticles. Nanoparticles enter senescent cells, where acidic conditions trigger liposomal or CaP dissolution. Senolytic agents such as dasatinib and quercetin induce apoptosis of senescent cells. Subsequently, NR and SRT2104 are released over time, restoring NAD⁺ levels and activating sirtuins. MSCs further promote regeneration by differentiation and secretion of paracrine factors.
Applications: The system may be used for prevention or treatment of age-related diseases such as osteoarthritis, sarcopenia, cardiovascular disease, metabolic dysfunction, neurodegeneration, and skin aging. In regenerative medicine, it may enhance outcomes in tissue engineering and wound healing. Personalized formulations may be achieved through autologous MSC integration and payload customization.
Supporting Data: Preliminary studies indicate ˜75% reduction in senescent cell burden, ˜2.5-fold elevation of NAD⁺ levels, and ˜3-fold increase in SIRT1 activity in treated cells. MSC engraftment rates reached ˜85% in target tissues. Controlled release studies confirmed ≤15% senolytic release at pH 7.4 and ≥80% release at pH 6.0 within 24 hours. These data are representative of preliminary in vitro and in vivo studies and are provided to illustrate feasibility. They are not intended as limiting or to imply clinical trial data.

### EXAMPLES

The following Examples illustrate certain embodiments of the invention and demonstrate feasibility through preliminary experiments. They are not intended to limit the scope of the invention, which is defined by the appended claims.

### Example 1: Synthesis of Calcium Phosphate Core Encapsulating NAD⁺ Precursor and Sirtuin Activator

A calcium phosphate nanoparticle core (100) was prepared by mixing CaCl₂ (100 mM) and Na₂HPO₄ (60 mM) at 0-5°C. with vigorous stirring. Nicotinamide riboside (110, 5 wt %) and SRT2104 (120, 3 wt %) were co-precipitated during nucleation. Resulting particles exhibited an average diameter of 110 nm with a polydispersity index of 0.14. Encapsulation efficiency was ˜65%.

### Example 2: Assembly of Liposomal Intermediate Layer With Senolytic Loading

The CaP cores were hydrated with a lipid film composed of phosphatidylcholine, dioleoylphosphatidylethanolamine (DOPE), and cholesteryl hemisuccinate (60:20:20 mol %). The suspension was extruded through 100 nm membranes. Dasatinib and quercetin (140, 15 wt %) were loaded into the lipid shell. Entrapment efficiency reached ˜70%.

### Example 3: Outer Iron Oxide Shell Deposition

Superparamagnetic Fe₃O₄ nanoparticles (5-10 nm) were adsorbed to the lipid shell to yield an outer layer (150) at 5 wt %. Magnetic responsiveness was confirmed by rapid magnetic separation (<60 seconds under 0.3 T field).

### Example 4: pH-Responsive Release Study

Nanoparticles were incubated at 37° C. in PBS buffers of pH 7.4, 6.8, and 6.0. Payload release was quantified by HPLC. At 24 h, RA24 was 82% at pH 6.0 and 70% at pH 6.8, while RN24 was 9% at pH 7.4. This confirms selective acidic release.

### Example 5: MSC Conjugation and Viability

PEG-azide modified nanoparticles were conjugated to alkyne-functionalized mesenchymal stem cells (200). Particle-to-cell ratios were 10³-10⁵. Flow cytometry confirmed ˜85% conjugation efficiency. MSC viability remained >90%.

### Example 6: In Vitro Efficacy

Doxorubicin-induced senescent fibroblasts treated with nanoparticles exhibited a 2.2-fold increase in NAD⁺ levels (400), a 2.1-fold increase in SIRT1 activity (410), and a 55% reduction in senescent cell burden (500).

### Example 7: Simulation-Guided Optimization

Simulations explored parameter space for particle size, Ca:P ratio, crystallinity, shell thickness, PDI, Fe₃O₄ content, and release kinetics. Optimal ranges identified include: 110±15 nm particle diameter, Ca: P 1.62±0.05, shell fraction 0.30±0.05, Fe₃O₄ 5±1 wt %, PDI≤0.15, RA24≥0.80 at pH≤6.5, RN24≤0.10 at pH 7.4. These results guided final design parameters for development lots.

1. A multi-layer nanoparticle system comprising: a biodegradable polymeric or calcium phosphate core encapsulating a nicotinamide riboside compound and a sirtuin activator; a pH-sensitive liposomal layer surrounding the core and encapsulating at least one senolytic agent; and an outer coating comprising magnetic nanoparticles for targeting and imaging.

2. A method of treating or delaying aging-related cellular dysfunction in a subject, comprising administering an effective amount of the multi-layer nanoparticle system of claim 1, wherein the system is configured to restore intracellular NAD⁺ levels, activate sirtuins, and selectively eliminate senescent cells.

3. A therapeutic composition comprising autologous mesenchymal stem cells, wherein the mesenchymal stem cells are surface-modified with a biocompatible coating and reversibly attached to a plurality of nanoparticles according to claim 1.

4. The system of claim 1, wherein the core comprises PLGA.

5. The system of claim 1, wherein the core comprises calcium phosphate nanoparticles.

6. The system of claim 1, wherein the sirtuin activator is SRT2104.

7. The system of claim 1, wherein the nicotinamide riboside is released in a sustained manner over at least 48 hours.

8. The system of claim 1, wherein the senolytic agent comprises dasatinib.

9. The system of claim 1, wherein the senolytic agent comprises quercetin.

10. The system of claim 1, wherein the liposomal layer destabilizes at a pH of about 6.5 or lower.

11. The system of claim 1, wherein the magnetic nanoparticles comprise superparamagnetic iron oxide nanoparticles.

12. The system of claim 1, wherein the magnetic nanoparticles enable guidance by an external magnetic field.

13. The system of claim 1, wherein the outer coating provides MRI contrast.

14. The method of claim 2, wherein administration is intravenous.

15. The method of claim 2, wherein senescent cells are selectively eliminated in an acidic microenvironment.

16. The method of claim 2, wherein NAD⁺ levels in target tissues increase by at least two-fold relative to baseline.

17. The composition of claim 3, wherein the coating comprises a hydrogel selected from alginate or PEG.

18. The composition of claim 3, wherein the nanoparticles are attached via a reversible click-chemistry linkage.

19. The composition of claim 3, wherein the mesenchymal stem cells are autologous.

20. The composition of claim 3, wherein the mesenchymal stem cells home to inflamed or damaged tissue.
